(19) United States
(12) Patent Application Publication
(10) Pub. No.: US 2026/0224613 A1
(43) Pub. Date: Aug. 6, 2026

(54) Use Of Inorganic Nitrate For Preventing Restenosis And/Or Device Thrombosis

(71) Applicant: Queen Mary University of London, London (GB)

(72) Inventors: Amrita Ahluwalia, London (GB); Anthony Mathur, London (GB)

(21) Appl. No.: 19/152,132

(22) PCT Filed: Jan. 31, 2024
(86) PCT No.: PCT/EP24/52420

§ 371 Date: Jul. 30, 2025

(30) Foreign Application Priority Data
Jan. 31, 2023 (GB) 2301354.3

(51) Int. Cl. A61K 33/00; A61K 36/21; A61K 45/06; A61P 9/10; A61P 9/14

(52) U.S. Cl. CPC A61K 33/00; A61K 36/21; A61K 45/06; A61P 9/10; A61P 9/14

(57) ABSTRACT

The present invention provides inorganic nitrate for use in a method of preventing restenosis and/or device thrombosis in a patient undergoing percutaneous coronary intervention (PCI), non-coronary percutaneous intervention and/or coronary artery bypass grafting (CABG), said method comprising administering to said patient 2-12 mmol of an inorganic nitrate once per day after said PCI, non-coronary percutaneous intervention and/or CABG.

### FIELD OF THE INVENTION

The present invention relates to the use of inorganic nitrate in a method of preventing restenosis and/or device thrombosis in a patient undergoing percutaneous coronary intervention (PCI), non-coronary percutaneous intervention and/or coronary artery bypass grafting (CABG).

### BACKGROUND TO THE INVENTION

Coronary heart disease is the single most common cause of death in the UK causing 1 in 7 and 1 in 10 deaths in men and women, respectively. Presently, timely percutaneous coronary intervention (PCI) with stent implantation remains the most effective treatment strategy for limiting events, infarct size following an acute myocardial infarction (AMI), preserving left ventricular ejection fraction, and improving clinical outcomes. However, despite these advances, substantial mortality and morbidity rates persist with respect to longer term outcomes.

In simple lesions, restenosis rates have been estimated to be less than 5% at 1 year but at 5 years, repeat intervention rates are approximately 10% (Weisz G, Leon M B, Holmes D R, Jr., Kereiakes D J, Popma J J, Teirstein P S, et al. J Am Coll Cardiol. 2009; 53 (17): 1488-97). However, in more complex lesions, restenosis has been documented at 10% within 2 years (Steinberg D H, Gaglia M A, Jr., Pinto Slottow T L, Roy P, Bonello L, De Labriolle A, et al. Am J Cardiol. 2009; 103 (4): 491-5.). In addition, recent assessments of patients undergoing PCI (~50% with stable angina) demonstrate that in-stent thrombosis, despite anti-platelet therapy, remains a concern, with recent calls urging identification of more effective and safer anti-platelet therapy (Kastrati A, Mehilli J, von Beckerath N, Dibra A, Hausleiter J, Pache J, et al. Jama. 2005; 293 (2): 165-71). A major determinant of prognosis after treatment is the reocclusion of the affected arteries. A number of specific phenomena have been linked with reocclusion including persistent endothelial dysfunction, increased platelet reactivity and restenosis.

In the healthy cardiovascular system, tonically generated NO, produced via the conventional L-arginine/NO synthesis pathway, plays an essential role in maintaining homeostasis and in sustaining healthy cardiac function, perfusion and cardioprotection (Bredt D S. Nitric oxide signaling specificity—the heart of the problem. J Cell Sci. 2003; 116 (Pt 1): 9-15; Moncada S, Higgs A. The L-arginine-nitric oxide pathway. N Engl J Med. 1993; 329 (27): 2002-12). In patients with coronary artery disease a generalised ‘endothelial dysfunction’ which is characterised by deficient endothelium-derived bioavailable NO exists; the extent of which is correlated with severity of coronary artery disease. The cardioprotective effects of NO relate to a number of actions including its potent vasodilator effect in the ischaemic myocardium, allowing for essential perfusion of injured tissue, its anti-inflammatory effects repressing leukocyte recruitment, its anti-platelet effects and its anti-proliferative influence over vascular smooth muscle.

A potential solution for elevating endogenous NO levels lies in the chemical reduction of inorganic nitrite (NO₂⁻) to NO. Indeed, nitrite-derived NO protects against myocardial ischaemia/reperfusion injury in pre-clinical models, attenuates vascular smooth muscle cell proliferation in a model of balloon injury in rats, protects against endothelial dysfunction and attenuates platelet reactivity in healthy volunteers and patients with hypercholesterolaemia. Importantly, inorganic nitrite does not suffer tachyphylaxis and its function is not dependent on metabolising enzymes that are dysfunctional in cardiovascular disease as with organic nitrates; factors that are major limiting issues underlying the difficulties with organic nitrates experienced within the clinical setting (Mayer B, Beretta M. BrJ Pharmacol. 2008; 155 (2): 170-84). However, it is well accepted that raising circulating levels of inorganic NO₂⁻ with a NO₂⁻ (nitrite) salt is impacted by issues of toxicity (Pluta R M, Dejam A, Grimes G, Gladwin M T, Oldfield E H. JAMA. 2005; 293 (12): 1477-1484. doi: 10.1001/jama.293.12.1477).

In summary, substantial mortality and morbidity rates persist with respect to longer term outcome in patients undergoing percutaneous coronary intervention (PCI). There is therefore a need in the art for effective preventative treatments for restenosis and device thrombosis in such patients.

### SUMMARY OF THE INVENTION

The present inventors have surprisingly found that inorganic nitrate, provided in the form of a dietary supplement, is effective in preventing restenosis and/or device thrombosis in patients undergoing percutaneous coronary intervention (PCI). In particular, it has been found that the administration of inorganic nitrate has a surprising effect on reducing major adverse cardiac events (MACE) in such patients.

Accordingly, in a first aspect the present invention provides inorganic nitrate for use in a method of preventing restenosis and/or device thrombosis in a patient undergoing percutaneous coronary intervention (PCI), non-coronary percutaneous intervention and/or coronary artery bypass grafting (CABG), said method comprising administering to said patient 2-12 mmol of an inorganic nitrate once per day after said PCI, non-coronary percutaneous intervention and/or CABG.

### DETAILED DESCRIPTION OF THE INVENTION

The present invention relates to inorganic nitrate (NO₃⁻) for use in a method of preventing restenosis and/or device thrombosis in a patient undergoing percutaneous coronary intervention (PCI), non-coronary percutaneous intervention and/or coronary artery bypass grafting (CABG).

Restenosis occurs when an artery which was previously opened up with a stent or angioplasty becomes narrowed again. In-stent restenosis refers to a blockage or narrowing in the portion of a blood vessel that has previously been opened up with a stent.

Thrombosis is a blood clot which blocks a blood vessel. Device thrombosis is typically stent thrombosis, i.e. thrombosis associated with the insertion of a stent.

By “preventing restenosis and/or device thrombosis” is meant at least partially avoiding the incidence of restenosis and/or device thrombosis. “Preventing” in the context of the present invention means either that restenosis and/or device thrombosis is completely prevented or the incidence of restenosis and/or device thrombosis is lower than would otherwise have been the case (without the use of inorganic nitrate).

The patient is typically a human patient, but the present invention also finds use in the veterinary field. The patient may therefore be a non-human mammalian patient, such as a cat, dog or other companion animal.

The present invention finds use in patients undergoing percutaneous coronary intervention (PCI), non-coronary percutaneous intervention and/or coronary artery bypass grafting (CABG).

Percutaneous coronary intervention (PCI) refers to a family of minimally invasive procedures used to open clogged coronary arteries (those that deliver blood to the heart). PCI may involve stent/scaffold/device implantation. PCI was formerly known as angioplasty with stent and is typically a non-surgical procedure that uses a catheter to place a stent in blood vessels in order to open them up. By restoring blood flow, the treatment can improve symptoms of blocked arteries, such as chest pain or shortness of breath.

Percutaneous intervention is also used for the treatment of narrowed arteries around the body e.g in the legs where this restriction of blood flow leads to symptoms of pain and sometimes loss of the limb. The invention therefore also finds use in patients undergoing non-coronary percutaneous intervention, requiring balloon and/or stent/scaffold combination e.g. peripheral vascular disease. Such interventions may also lead to restenosis. The invention can therefore also be applied to patients with peripheral vascular disease requiring percutaneous intervention (e.g. angioplasty) e.g. limb ischaemia.

Coronary artery bypass grafting (CABG) is a surgical procedure used to treat coronary heart disease and diverts blood around narrowed or clogged parts of the major arteries to improve blood flow and oxygen supply to the heart. The procedure involves taking a blood vessel from another part of the body (usually the chest, leg or arm) and attaching it to the coronary artery above and below the narrowed area or blockage. CABG is also referred to as post-bypass grafting.

Inorganic nitrate is used in the present invention. Inorganic nitrate is a polyatomic ion naturally found in a range of foods such as beetroot and green leafy vegetables and is also added in the form of nitrate salts (such as potassium nitrate and sodium nitrate) as a preservative to processed meat products such as ham and bacon.

Inorganic nitrate may be administered in the form of dietary nitrate. Dietary nitrate may be in the form of any nitrate-rich vegetable or fruit, for example beetroot, green leafy vegetables (such as spinach, cabbage, Chinese cabbage and lettuce), fennel, rocket, radish and parsley, or a juice or dried concentrate thereof. The dietary nitrate may be administered in the form of beetroot juice or the juice of any nitrate-rich vegetable.

Alternatively, the inorganic nitrate may be administered in the form of a nitrate salt, for example potassium nitrate (KNO₃), sodium nitrate (NaNO₃), calcium nitrate (Ca(NO₃)₂), zinc nitrate (Zn(NO₃)₂) or ammonium nitrate (NH₄NO₃).

The inorganic nitrate may be administered in the form of a preparation or pharmaceutical composition, including any pharmaceutically acceptable excipient, diluent or binder.

The inorganic nitrate is typically administered orally. Alternatively, the inorganic nitrate may be administered parenterally (sub-cutaneously, intravenously, via suppository, as a patch etc).

For oral administration, the inorganic nitrate may be administered in the form of a capsule, pill, lozenge, spray, toothpaste or chewing gum.

The dosage of inorganic nitrate for use in the present invention is from 2-12 mmol once per day, typically 2.5-8 mmol once per day. For example, 2-11 mmol, 2.5-10 mmol, 2.5-9 mmol, 3-7 mmol, 4-6 mmol or 4-5 mmol, for example 4 mmol or 5 mmol of nitrate may be administered once per day.

Administration of the inorganic nitrate may begin after the patient has undergone a PCI, non-coronary percutaneous intervention and/or CABG. Alternatively, the inorganic nitrate may be administered prior to said PCI, non-coronary percutaneous intervention and/or CABG. In this instance, the inorganic nitrate may be administered at least 2 hours prior to said PCI, non-coronary percutaneous intervention and/or CABG. Typically, the inorganic nitrate is administered no more than 24 hours prior to said PCI, non-coronary percutaneous intervention and/or CABG. Accordingly, the inorganic nitrate may be administered from 2-24 hours prior to said PCI, non-coronary percutaneous intervention and/or CABG, for example from 3-22 hours, 4-21 hours, 5-20 hours, 6-19 hours, 7-18 hours, 8-17 hours, 9-16 hours, 10-15 hours, 11-14 hours, 12-13 hours prior to said PCI, non-coronary percutaneous intervention and/or CABG.

Administration of the inorganic nitrate may continue on a once daily basis for 1-5 months after the patient has undergone a PCI, non-coronary percutaneous intervention and/or CABG. For example, administration of the inorganic nitrate may continue on a once daily basis for 1 month, 2 months, 3 months, 4 months or 5 months after the patient has undergone a PCI, non-coronary percutaneous intervention and/or CABG or up to 6 months after the patient has undergone a PCI, non-coronary percutaneous intervention and/or CABG. Alternatively, administration of the inorganic nitrate may continue on a once daily basis for at least 6 months after the patient has undergone a PCI, non-coronary percutaneous intervention and/or CABG. For example, administration of the inorganic nitrate may continue on a once daily basis for at least 7 months, at least 8 months, at least 9 months, at least 10 months, at least 11 months, at least 12 months, at least 14 months, at least 16 months, at least 18 months or at least 2 years, 3 years, 4 years, 5 years, 6 years, 7 years, 8 years, 9 years or 10 years. The inorganic nitrate may be administered on a once daily basis indefinitely, for the remainder of the patient's life.

The inorganic nitrate may be administered in combination with an antioxidant.

Inorganic nitrite may be administered from a coating applied to a stent or scaffold or prosthesis used in percutaneous intervention (coronary or non-coronary).

The patient undergoing percutaneous coronary intervention (PCI), non-coronary percutaneous intervention and/or coronary artery bypass grafting (CABG) is typically suffering from one or more medical conditions. For example, the patient may have angina, for example stable (chronic) angina. Angina is chest pain caused by reduced blood flow to the heart muscles. Stable angina is more common and less serious than unstable angina. Attacks typically have a trigger, for example stress or exercise, and usually stop within a few minutes of resting. Alternatively, the patient may have an acute coronary syndrome (ACS). Acute coronary syndromes include a range of conditions associated with sudden, reduced blood flow to the heart, for example unstable angina or myocardial infarction. Unstable angina is less common and more serious than stable angina. Attacks are more unpredictable and represent a medical emergency. Myocardial infarction (MI), acute myocardial infarction (AMI) or heart attack is a serious medical emergency and occurs when blood flow to the heart is suddenly blocked. The patient may in addition be suffering from diabetes, either type I or type II, and/or peripheral vascular disease.

The present inventors have surprisingly found that administration of inorganic nitrate reduces the incidence of major adverse cardiac events (MACE) in patients undergoing percutaneous coronary intervention (PCI), non-coronary percutaneous intervention and/or coronary artery bypass grafting (CABG). Accordingly, the present invention may alternatively be worded as inorganic nitrate for use in a method of reducing the incidence of or preventing MACE in a patient undergoing percutaneous coronary intervention (PCI), non-coronary percutaneous intervention and/or coronary artery bypass grafting (CABG), said method comprising administering to said patient 2-12 mmol, typically 2.5-8 mmol, of an inorganic nitrate once per day after said PCI, non-coronary percutaneous intervention and/or CABG.

The present invention can also be worded as:
A method of preventing restenosis and/or device thrombosis in a patient undergoing percutaneous coronary intervention (PCI), non-coronary percutaneous intervention and/or coronary artery bypass grafting (CABG), said method comprising administering to said patient 2-12 mmol, typically 2.5-8 mmol, of an inorganic nitrate once per day after said PCI, non-coronary percutaneous intervention and/or CABG.
A method of reducing the incidence of or preventing MACE in a patient undergoing percutaneous coronary intervention (PCI), non-coronary percutaneous intervention and/or coronary artery bypass grafting (CABG), said method comprising administering to said patient 2-12 mmol, typically 2.5-8 mmol, of an inorganic nitrate once per day after said PCI, non-coronary percutaneous intervention and/or CABG.

Preferred features for the various aspects of the invention are as for the first aspect of the invention mutatis mutandis. It will be appreciated that all embodiments described herein are considered to be broadly applicable and combinable with any and all other consistent embodiments, as appropriate. Such combinations are considered to fall within the scope of the present invention.

### BRIEF DESCRIPTION OF FIGURES

FIG. 1—The two main pathways for NO synthesis in the body: the canonical and the non-canonical. The present invention focuses on utilizing the non-canonical pathway to deliver NO. After ingestion of oral inorganic NO₃⁻ it is swallowed and enters the GI tract, where it is then absorbed and accumulates in the circulation. Approximately 75% of ingested NO₃⁻ is excreted via the kidneys and the remaining 25% is concentrated in salivary glands. This NO₃⁻ enters the saliva where it is reduced to NO₂⁻ by oral bacteria. NO₂⁻ is then swallowed thereby entering the GI tract and subsequently resulting in the accumulation of NO₂⁻ in the circulation. Once within the circulation, NO₂⁻ is reduced to NO in the vasculature where it then exerts a range of positive effects (vasodilatation, cytoprotective, anti-inflammatory and anti-thrombotic) shown in the figure via the downward arrows.

FIG. 2—Effects of NaNO₂ on cell proliferation of Human Coronary Arterial Endothelial Cells (HCAEC) and Human Coronary Arterial Smooth Muscle Cells (HCSMC) exposed to Ischaemia/Reperfusion injury. Cell Count of HCAEC and HCASMC respectively under hypoxia (0.1% O₂, 24 h) and re-oxygenation (normoxia, 8, 24 and 48 h post ischaemia)) in the absence and presence of NaNO₂ 3 μM. n=7/group for HCAEC and n=5/group for HCASMC. Results are expressed as % changes of the respective matched normoxia controls. Data represent mean±SEM of the number of independent experiments performed as indicated above. Statistical significance determined using two-way ANOVA followed by post-test comparing response at each timepoint between treatments determined using Sidak's multiple comparison test.

FIG. 3—Angiogram images of the left anterior descending artery (LAD). LAD (indicated by the white arrow) A) at baseline before the stent was implanted, B) at baseline after the stent and C) at 6 months after the stent.

FIG. 4—Consort Diagram for Nitrate OCT study.

FIG. 5—Dietary nitrate prevents restenosis. (A) In-stent late lumen loss and (B) In-segment late lumen loss at 6 months are reduced following inorganic nitrate treatment versus the placebo. Values are mean±SEM and statistical significance is demonstrated using an unpaired t-test.

FIG. 6—Major Adverse Cardiac Events (MACE) over 2 years. MACE over 2 years was demonstrated between the two groups using intention to treat analysis. The statistical significance is demonstrated using a Log-Rank test. Intervention: dietary nitrate via nitrate-containing beetroot juice. Placebo: nitrate-depleted beetroot juice.

FIG. 7—Major Adverse Cardiac Events (MACE) over 2 years. MACE over 2 years was demonstrated between the two groups using per protocol analysis (excluding individuals receiving a bare metal stent). The statistical significance is demonstrated using a Log-Rank test. Intervention: dietary nitrate via nitrate-containing beetroot juice. Placebo: nitrate-depleted beetroot juice.

### EXAMPLES

### Example 1—Nitrate OCT Study to Assess the Safety and Efficacy of Oral Nitrate in a Dietary Form

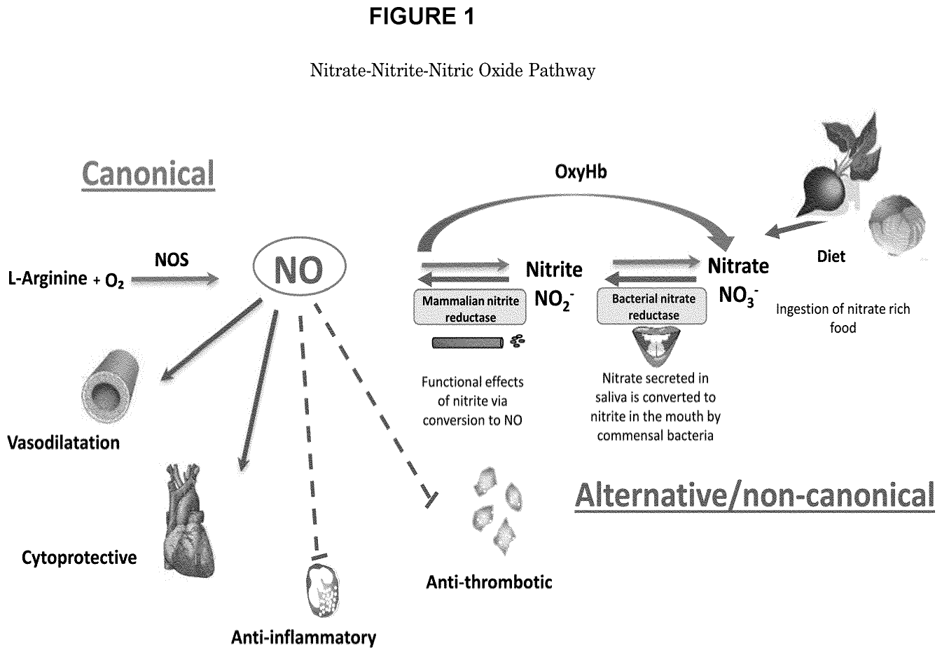

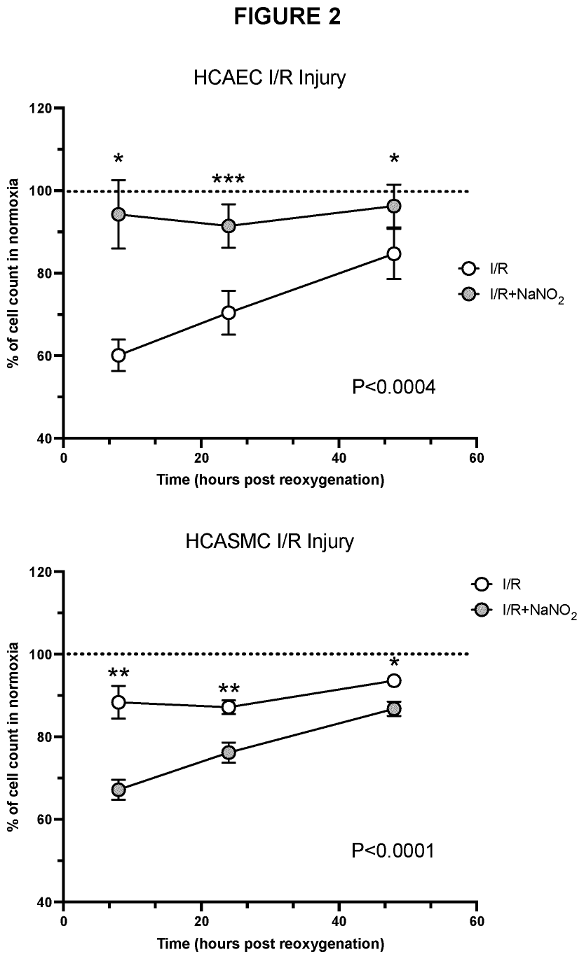

A key method for delivery of inorganic nitrite to the body is via the administration of dietary nitrate. This nitrate enters the body where it is converted in the oral cavity by bacteria to nitrite and then this nitrate enters the circulation where it is converted to NO (FIG. 1). The inventors suggested that elevating nitrite might be useful for attenuating restenosis since they have previously shown anti-platelet effects of the non-canonical pathway and have unpublished findings (FIG. 2) demonstrating that nitrite improves endothelial cell growth following an ischaemia-reperfusion insult whilst simultaneously suppressing the growth of vascular smooth muscle cells (a key factor in restenosis development).

There have been no clinical studies investigating the role of orally ingested nitrate in reducing restenosis in patients undergoing percutaneous coronary intervention (PCI) for stable angina. NITRATE-OCT (OCT: optical coherence tomography) study is the first clinical study assessing the safety and efficacy of oral nitrate in a dietary form (beetroot juice) in this group of patients.

This study is designed to test whether dietary nitrate ingestion in addition to conventional therapy might exert beneficial effects in individuals susceptible to serious cardiac events. In particular, the aim is to investigate whether dietary nitrate might exert anti-platelet effects, improvement of endothelial function and improvements of intimal hyperplasia thereby possibly resulting in reductions of restenosis rates post PCI and stent implantation.

Specifically the aims are:

1) To determine whether dietary nitrate might improve intimal hyperplasia and thereby restenosis rates post PCI and stent implantation.

2) To determine whether dietary nitrate ingestion exerts anti-platelet effects or improvement of endothelial function and the mechanisms involved in this effect.

NITRATE-OCT is a double-blind, randomised, single-centre, placebo-controlled phase II trial that enrolled 300 patients with stable angina due to have an elective percutaneous coronary intervention (PCI) procedure with stent implantation. Patients were randomised to receive 6 months of a once-a-day dose of either nitrate-rich beetroot juice or nitrate-deplete beetroot juice (placebo) starting up to one week before their procedure. The primary powered outcome was reduction of in-stent late-loss assessed by angiography at 6 months and was also compared with assessment using optical coherence tomography (OCT) at 6 months. Secondary endpoints also include change from baseline assessment of endothelial function measured using ultrasound for assessment of flow-mediated dilatation of the brachial artery at 6 months, target vessel revascularisation (TVR), restenosis rate (diameter>50%) and in-segment late loss at 6 months, markers of inflammation and platelet reactivity and major adverse cardiac events (i.e. myocardial infarction, death, cerebrovascular accident, TVR) at 12 and 24 months. FIG. 1 shows a schematic of the study.

A total of 300 patients (male and female, age 18-85) with stable angina as per requirements indicated below were recruited. Since patients who are diabetics are at high risk of developing restenosis, these patients were also included in our study with stratification in both groups. Patients were stratified according to the type of stent (i.e. bare metal stent or drug eluting stent) as both these groups have differing characteristics resulting in differing rates of restenosis. Follow-up took place in the Clinical Trials Centre at The William Harvey Heart Centre. The patients inevitably had other co-morbidities that included raised blood pressure and hypercholesterolemia, and these were recorded. Patients were block randomised (using an online randomisation database) to receive 70 ml of a beetroot juice concentrate containing ~5 mmol nitrate or nitrate-depleted placebo juice concentrate (James White Drinks) control. The volunteers commenced taking their daily dose at home the day before the scheduled angioplasty and continued daily for 6 months. Patients were advised to take their dose of juice at the same time each day, preferably in the morning with their breakfast. Patients were also provided with dietary advice in relation to the calorific content of the juice: a daily dose of 70 ml of the juice concentrate (approximately 70 g) contains about 100 kcal.

### Primary Endpoint:

The primary endpoint was reduction of in-stent late loss assessed by angiography (quantitative coronary angiography, QCA) at 6±1 months. Assessment of restenosis was also made by measurement of in-stent late-loss using optical coherence tomography (OCT) at 6±1 month.

### Secondary Endpoints:

(i) Improvement in endothelial function assessed by flow-mediated dilatation (FMD) of the brachial artery at 6 months compared to pre-procedure assessment.
(ii) Reduction in target vessel revascularisation (TVR), restenosis rate (diameter>50%) and in-segment late loss at 6±1 month.
(iii) Reduction in major adverse cardiac events (i.e. MI, death, CVA, TVR) at 6, 12 and 24 months
(iv) Reduction in plaque size as assessed using OCT at 6±1 month.
(v) Reduction in inflammatory markers and changes in plasma xanthine oxidoreductase (XOR) activity, hsCRP, IL-6 at 6 and 12 months.
(vi) Reduction in platelet aggregation ex vivo at 6 and 12 months compared to pre-procedure.

### Study Inclusion Criteria

1. Patients with stable angina diagnosed by a cardiologist on optimal medical therapy undergoing angioplasty to treat residual symptoms.
2. Aged 18-85
3. Patients able and willing to give their written informed consent.
4. Patients undergoing successful PCI procedure.

### Study Exclusion Criteria

1. Unstable ischaemic heart disease, with an episode of chest pain in less than 24 hours before inclusion into the study.
2. Patients who have had previous coronary artery bypass surgery (CABG), if they are undergoing angioplasty within a non-native vessel.
3. Patients undergoing angioplasty with a bio-absorbable stent.
4. Current diagnosis of, or treatment for, malignancy other than non-melanoma skin cancer.
5. Current life-threatening condition other than vascular disease that may prevent a subject completing the study.
6. Use of an investigational device or investigational drug within 30 days or 5 half-lives (whichever is the longer) preceding the first dose of study medication.
7. Patients considered unsuitable to participate by the research team (e.g. due to medical reasons, laboratory abnormalities, or subject's unwillingness to comply with all study related procedures).
8. Severe acute infection, or significant trauma (burns, fractures).
9. Pregnancy tested by urine HcG measurement
10. History of alcohol or drug abuse within the past 6 months.
11. A history of heart failure NYHA class 3-4 or severe LV dysfunction LVEF<30% regardless of symptom status.
12. Systemic autoimmune disease such as rheumatoid arthritis, connective tissue disease, or other conditions known to be associated with chronic inflammation such as inflammatory bowel disease.
13. Patients who have donated >500 ml blood within 56 days prior to study medication administration.
14. Anaemia with Hb<10 g/dl, or any other known blood disorder or significant illness that may affect platelet function, and coagulation.
15. A history of chronic viral hepatitis (including presence of hepatitis B surface antigen or hepatitis C antibody or other chronic hepatic disorder) or HIV.
16. Abnormal liver function due to acute or chronic liver conditions 3×upper limit of normal at screening.
17. Renal impairment with creatinine clearance (eGFR) of 35 ml/min at screening.
18. If patients are on mouthwash, they must be willing to stop using this at least 1 week before the start of the study and throughout the duration that they are involved in the study.

### Randomisation and Blinding Process

Patients were block randomised on a 1:1 basis to receive either dietary nitrate or placebo, using a binary random number sequence (www.random.org). Treatment assignment for volunteers in both the dietary nitrate and placebo groups remained blinded until data lock and statistical analysis at the end of the study. If un-blinding was required, a list of the un-blinded treatments were kept in a secure location at The William Harvey Heart Centre. The un-blinding procedure was available at all times (24 hrs day/7 days a week).

### Blood, Saliva and Urine Analysis

In this study, blood samples were taken for the assessment of platelet reactivity, and nitric oxide level determination from the venous side of the circulation from an arm vein using a yellow butterfly needle (19 gauge). In this study protocol on visit two, they had one such blood test before starting the treatment or placebo juice. They then had a further blood test at visit three (6 months) and visit five (12 months). Blood samples were centrifuged immediately for platelet reactivity experiments and plasma and red blood cell storage only for the purposes of making biochemical measurements (e.g. nitrate/nitrite/cGMP) and were discarded once used.

Saliva and urine was collected in a falcon tube at the time of visits and stored for purposes of measuring nitrate/nitrite and then discarded. Some saliva was centrifuged and a pellet generated. This pellet contained oral bacteria that had dislodged from the oral cavity. This pellet was frozen for identification and analysis of the oral microbiota by second generation genome sequencing.

The nitrate/nitrite concentrations in saliva, blood and urine were determined using the technique of chemiluminescence as previously described in Ignarro L J, Fukuto J M, Griscavage J M, Rogers N E, Byrns R E. Proc Natl Acad Sci USA. 1993; 90 (17): 8103-7. In brief, total nitrate and nitrite concentration (termed “NOx”) was determined by adding samples to 0.1 mol/L vanadium (III) chloride in 1 M hydrochloric acid refluxing at 95° C. under nitrogen. Nitrite concentration was determined by the addition of samples to 0.09 mol/L potassium iodide in a glacial acetic acid under nitrogen at room temperature. Nitrate concentration was calculated by the subtraction of [nitrite] from [NOx]. All measurements of all samples were conducted by an individual blinded to the intervention.

### Pulse Wave Analysis (PWA) and Pulse Wave Velocity (PWV)

This technique is a non-invasive method to measure arterial stiffness. A Vicorder device (Skidmore Medical Limited, Bristol, UK) was used to simultaneously record the pulse wave from the carotid and femoral site using an oscillometric method. A small, inflatable neck pad was placed directly over a single carotid artery and secured around the neck by a Velcro tab. A cuff was placed around the patient's ipsilateral upper thigh. Both carotid and femoral cuffs were inflated automatically to 65 mmHg and the corresponding oscillometric signal from each cuff was digitally analysed to extract the pulse time delay. The distance between the sternal notch and the thigh cuff was measured and used as a standard estimate for the aortic length. From these measurements aortic pulse wave velocity (PWV) can be derived as PWV=aortic distance/pulse time delay, as described in Hickson S S, Butlin M, Broad J, Avolio A P, Wilkinson I B, McEniery C M. Hypertension research: official journal of the Japanese Society of Hypertension. 2009; 32 (12): 1079-85.

These procedures were not uncomfortable and took approximately 10 minutes to complete and were conducted at visit two (baseline), visit three (6 months) and visit five (12 months).

### Index PCI

Procedural information for the index PCI included date of the PCI, which coronary vessels and segments were treated, diameter, length and type of stent implanted, the procedural success for each segment, the adjunctive pharmacological therapy and any complications and information on lesions not treated were also recorded.

Repeat Coronary Angiography with OCT Assessment of Vessel Treated PCI

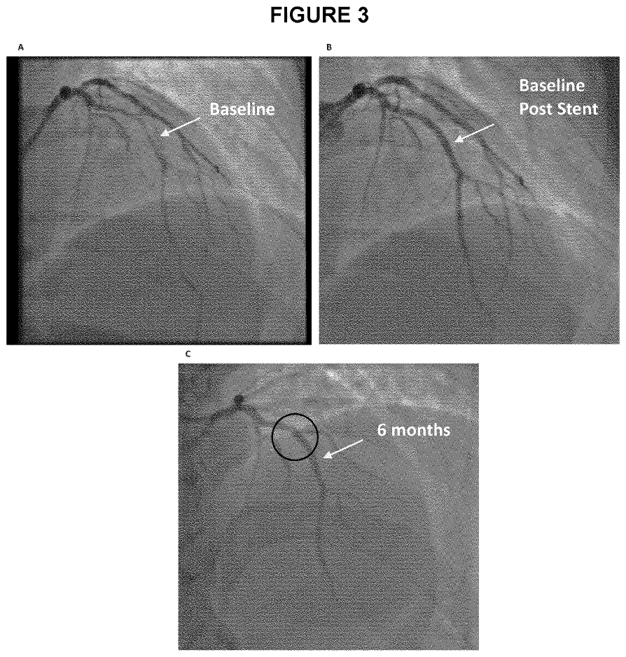

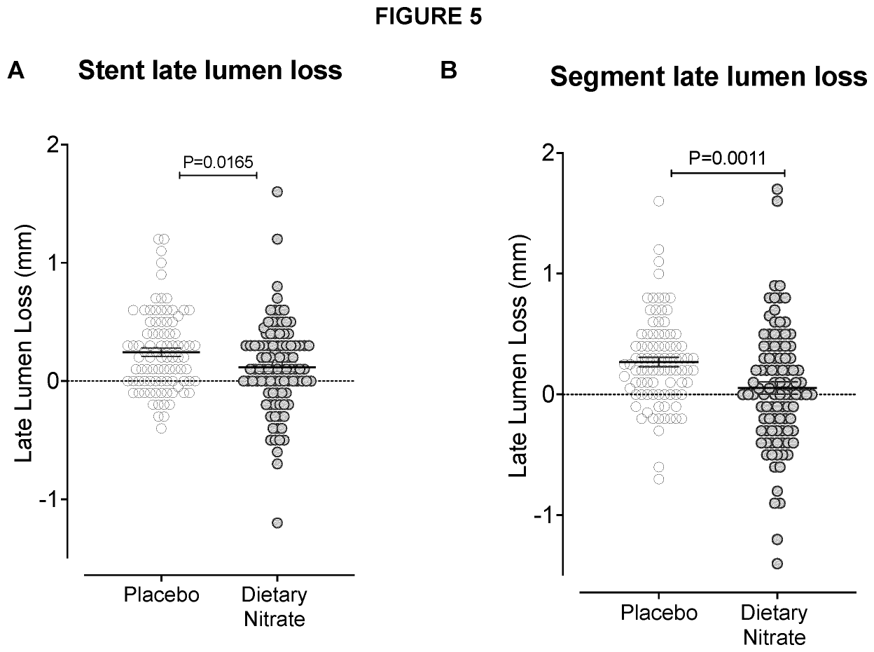

Repeat angiography and OCT were conducted at 6±1 month after the index PCI. Either radial or femoral access sites were used and angiograms of the involved vessel performed in at least 2 near orthogonal views. Quantitative angiographic assessment of the images gained through this method (FIG. 3) involved vessels were undertaken to determine late loss. Results are shown in FIG. 5.

### Follow Up

After 2 years, the participant was contacted by telephone for assessment of major adverse cardiac events (MACE). The whole duration of the study was 2 years. Participants had no obligation to complete the whole study and if they decided to withdraw at any point then they were free to do so.

The schedule of assessment is shown in Table 1 below.

| | | | | Visit 3 | Visit 4 | Visit 5 | |
|---|---|---|---|---|---|---|---|
| | | Visit 2 | | 6 | Angiogram | 12 | |
| | | Baseline | | months | and OCT | months | |
| | Screening | (Prior to | During | post | 6 ± 1 month | post | 2 |
| Procedures | Visit 1 | PCI) | PCI | PCI | post PCI | PCI | years |
| Screening for | x | | | | | | |
| inclusion/exclusion | | | | | | | |
| criteria | | | | | | | |
| Physical exam | | x | | | | | |
| Consent | | x | | | | | |
| Randomisation | | x | | | | | |
| Bloods ¹ | | x | | x | | x | |
| Saliva ² | | x | | x | | x | |
| Urine ³ | | x | | x | | x | |
| PWA and PWV | | x | | x | | x | |
| Flow mediated | | x | | x | | | |
| dilatation | | | | | | | |
| Ingestion of | | x | x | x | | | |
| juice (Nitrate | | | | | | | |
| replete or | | | | | | | |
| nitrate deplete | | | | | | | |
| placebo) + | | | | | | | |
| completing a | | | | | | | |
| daily juice diary | | | | | | | |
| PCI | | | x | | | | |
| Angiogram and | | | | | x | | |
| OCT | | | | | | | |
| MACE follow-up | | | | x | | x | |
| Assessment for | | x | x | x | x | x | |
| adverse events | | | | | | | |
| and reactions | | | | | | | |
| Assessment for | | | | | | | x |
| adverse events | | | | | | | |
| and reactions | | | | | | | |
| via telephone | | | | | | | |
| call | | | | | | | |

Table 1—Schedule of Assessment. ¹—FBC, U&E, CRP, nitrite, nitrate, cGMP, inflammatory markers (chemokines including CRP, CXCL-5, CXCL-8, CCL-2, and facs analysis). Standard bloods were performed at local lab. Platelet function, markers of inflammation and nitrite levels measured at The William Harvey Research Institute. Assessments were conducted where sample were available at baseline, 6 and 12 months. Markers of inflammation (chemokines including CRP, CXCL-5, CXCL-8, CCL-2 by flow cytometry) were measured at baseline, 6 and 12 months where samples were available. Markers of platelet reactivity were measured at baseline, 6 and 12 months. ²—Saliva was collected in a falcon tube at the time of visits and stored for purposes of measuring nitrate/nitrite and then discarded. Some saliva was centrifuged and a pellet generated. This pellet contained oral bacteria that had dislodged from the oral cavity. This pellet was frozen for identification and analysis of the oral microbiota by second generation genome sequencing at a later date. ³—Blood, urine and saliva were used to measure levels of nitrite, nitrate and cGMP at baseline measured at baseline, 6 and 12 months. PWA: Pulse Wave Analysis. PWV: Pulse Wave Velocity. MACE: Major Adverse Cardiac Events. OCT: Optical Coherence Tomography.

### End of Study Definition

The study finished 2 years after the telephone follow-up of the last patient.

### Statistical Analysis

Sample Size: Sample size was determined for a total of 246 patients that entered a two treatment parallel-design study. The probability was 80 percent that the study would detect a treatment difference at a two sided 5% significance level, if the true difference in late loss between the treatments is 0.22 mm. This absolute difference was calculated from a mean late loss of 1.27 mm with a standard deviation of the response variable of 0.550. These values being the mean and average of the standard deviations of 22 trials measuring late loss in both drug eluting and bare metal stents described in Mauri L, Orav E J, Kuntz R E. Circulation. 2005; 111 (25): 3435-42. Recruitment also took into account an additional 10% to account for drop-out or withdrawal/non-compliance. This value was based upon previous experience in the unit of the inventors.

The sample size of 246 enabled sufficient power for estimation of the hard endpoint of MACE at 6 months. Very recently, in stable angina patients who have undergone elective angioplasty a remote ischaemic preconditioning intervention resulted in a significant reduction in MACE at 6 months with 4/110 (3.6%) in the treatment group versus 13/104 (12.5%) in the control group (Davies W R, Brown A J, Watson W, McCormick L M, West N E J, Dutka D P, et al. Remote Ischemic Preconditioning Improves Outcome at 6 Years After Elective Percutaneous Coronary Intervention: The CRISP Stent Trial Long-term Follow-up. Circulation: Cardiovascular Interventions. 2013; 6 (3): 246-51). Using these data as a basis for power calculations, a total number of 230 patients were needed for 80% power using one-tailed analysis. Thus, to account for potential loss to follow-up recruitment of 246 patients in total was achieved. Due to dropout an amendment was submitted to enable recruitment of 300 patients to achieve power for the primary outcome.

All statistical analysis was conducted by the trial statisticians supported by the Imperial Clinical trials unit (CTU) and at The William Harvey Research Institute. Data was analysed on an intention to treat basis. Further per protocol analyses and a subgroup analysis were conducted on patients who were on organic nitrates as part of their routine therapy and a comparison of drug-eluting stents (DES) versus bare-metal stents (BMS).

### Results

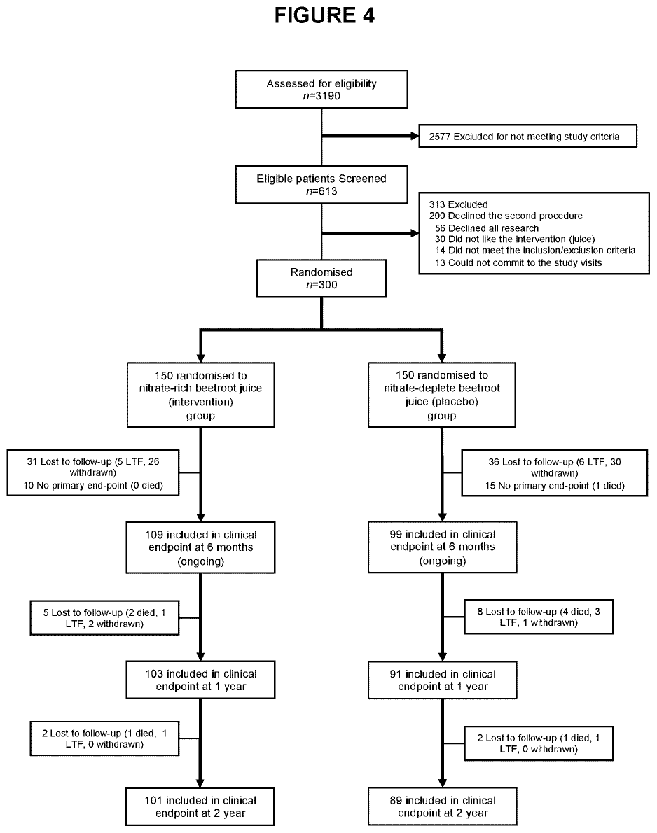

NITRATE-OCT enrolled 300 patients with angina and 150 were randomised to receive nitrate-containing beetroot juice (intervention group), and 150 to nitrate-depleted beetroot juice (placebo group) for 6 months (FIG. 4). The baseline demographic data of the two groups is similar indicating effective randomisation (Table 2). Similarly, the procedural characteristics of the index PCI were similar between the groups, with 69.3% and 74% of the patients received Xience drug-eluting stents in the nitrate and placebo groups, respectively (Table 3). The primary endpoint of stent late lumen loss in the patients treated with dietary nitrate was 0.117 mm+0.038 compared with 0.244 mm+0.036 in the patients treated with placebo (P=0.0165) (Table 4 and FIG. 5). In addition, segment late lumen loss was observed to be 0.055 mm+0.050 in the dietary nitrate group, and 0.269 mm+0.039 in patients treated with placebo (P=0.0011). These observations demonstrate clearly that dietary nitrate treatment prevented the loss of lumen over time following PCI in patients with stable angina.

This beneficial effect of dietary nitrate is likely due to the elevation of circulating nitrite and thus delivery of nitric oxide to the index vessel since dietary nitrate treatment resulted in an increase in the plasma NO₃⁻ concentration of approximately 6.14 fold and an increase in plasma NO₂⁻ of approximately 2.01 fold at 6 months compared to baseline where no elevations were evident in the placebo group. Confirmation of dietary nitrate efficacy is provided from the changes in haemodynamics. Systolic blood pressure (SBP) at 6 months compared to baseline was lower in the dietary nitrate treated group with a change of SBP of −11.99 mmHg (IQR: −18.00-4.50) compared to the placebo group (1.51 mmHg, IQR: −5.00-9.00, P<0.0001) (Table 5).

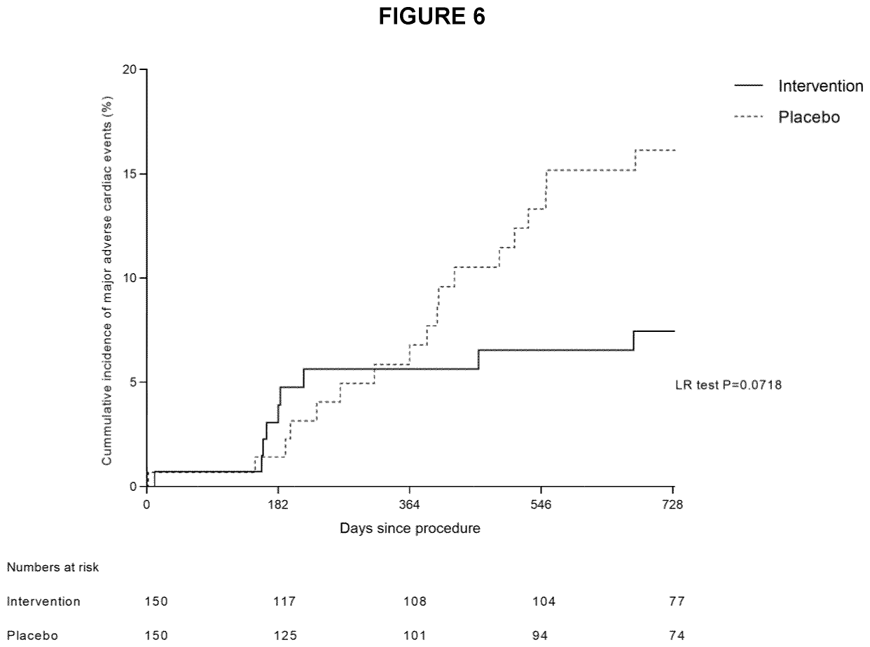

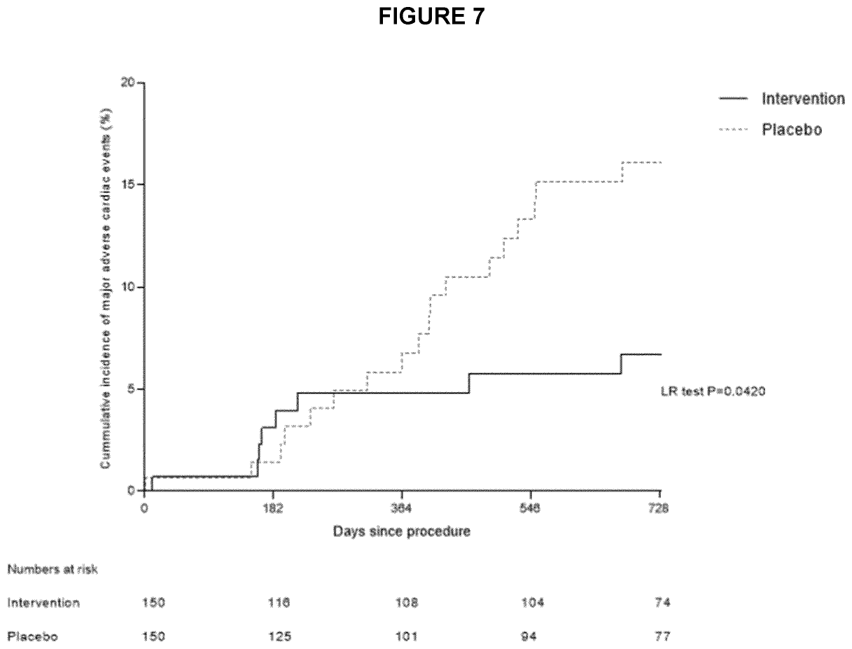

Whilst these effects above suggest positive influence over restenosis, the impact upon MACE was very surprising. At 2 years following the procedure in the whole cohort, the number of MACE events appeared to be substantially attenuated (FIG. 6). This effect did not quite reach statistical significance (P<0.0718). However, analysis of the data as per a prior sub-group assessment of those only receiving a DES indicated a substantial and statistically significant reduction in MACE in those patients consuming dietary nitrate (FIG. 7).

Interpretation: In patients who underwent PCI for stable coronary disease, treatment with dietary nitrate, there was a significant decrease in restenosis.

TABLE 2
Baseline characteristics of recruited patients
Table 2. Baseline characteristics

| | Intervention (n = 150) | | Placebo (n = 150) | | P value |
|---|---|---|---|---|---|
| Age (years) (mean ± SD) | 61.59 ± 9.79 | | 61.59 ± 8.88 | | 0.897 |
| Sex (Male) | 130 | (86.7%) | 131 | (87.3%) | 1.000 |
| Ethnicity | | | | | |
| Caucasian | 119 | (79.3%) | 120 | (80.0%) | 1.000 |
| Afro-Caribbean | 10 | (6.7%) | 10 | (6.7%) | 1.000 |
| Asian | 18 | (12%) | 18 | (12.0%) | 1.000 |
| East Asian | 2 | (1.3%) | 2 | (1.3%) | 1.000 |
| Diabetes mellitus | 33 | (22%) | 33 | (22.0%) | 1.000 |
| Type I | 1 | (0.7%) | 1 | (0.7%) | 1.000 |
| Type II | 32 | (21.3%) | 32 | (21.3%) | 1.000 |
| Body-mass index (kg/m²) | 28.81 ± 4.44 | | 28.95 ± 4.77 | | 0.727 |
| (mean ± SD) | | | | | |
| Hypertension | 110 | (73.3%) | 111 | (74.0%) | 0.896 |
| Hypercholesterolaemia | 104 | (69.3%) | 112 | (74.7%) | 0.304 |
| Previous MI | 55 | (36.7%) | 56 | (37.3%) | 1.000 |
| Previous PCI | 47 | (%) | 55 | (36.7%) | 0.394 |
| Previous CABG | 6 | (4.0%) | 8 | (5.3%) | 0.785 |
| Current Smoker | 22 | (%) | 33 | (22.0%) | 0.101 |
| Previous Smoker | 74 | (%) | 65 | (43.3%) | 0.297 |
| PVD | 11 | (%) | 10 | (6.7%) | 0.821 |
| CVA/TIA | 7 | (%) | 7 | (4.7%) | 0.100 |
| NYHA | | | | | |
| Class I | 6 | (4.0%) | 10 | (6.7%) | 0.442 |
| Class II | 8 | (5.3%) | 6 | (4.0%) | 0.785 |
| Class III | 0 | | 2 | (1.3%) | 0.498 |
| CCS | | | | | |
| CCS I | 28 | (18.7%) | 27 | (18.0%) | 1.000 |
| CCS II | 53 | (29.4%) | 52 | (34.7%) | 1.000 |
| CCS III | 66 | (44.0%) | 66 | (44.0%) | 1.000 |
| CCS IV | 1 | (0.7%) | 4 | (2.7%) | 0.371 |
| Asthma | 17 | (11.3%) | 13 | (8.7%) | 0.441 |
| COPD | 9 | (6.0%) | 9 | (6.0%) | 1.000 |
| Previous History of CAD | 75 | (50.0%) | 80 | (53.3%) | 0.563 |
| Heart rate (BPM) (mean ± SD) | 69.49 ± 13.78 | | 66.85 ± 12.40 | | 0.089 |
| Systolic BP (mmHg) (mean ± SD) | 137.18 ± 16.86 | | 136.79 ± 16.85 | | 0.845 |
| Diastolic BP (mmHg) (mean ± SD) | 78.97 ± 9.90 | | 77.43 ± 10.12 | | 0.199 |
| Culprit Vessel | | | | | |
| Left main stem | 0 | | 0 | | — |
| Left anterior descending | 55 | (36.7%) | 66 (44.0%) | | 0.168 |
| First diagonal | 5 | (3.3%) | 0 | | 0.530 |
| Intermediate Artery | 2 | (1.3%) | 3 | (2.0%) | 0.550 |
| Circumflex | 26 | (17.3%) | 19 | (12.7%) | 0.374 |
| Obtuse Marginal | 4 | (2.7%) | 4 | (2.7%) | 0.620 |
| Right coronary | 46 | (30.7%) | 41 | (27.3%) | 0.563 |

TABLE 3
Procedural characteristics of the PCI
Table 3. Procedural characteristics

| | Intervention (n = 150) | | Placebo (n = 300) | | P value |
|---|---|---|---|---|---|
| Radial access | 124 | (82.7%) | 121 | (80.7%) | 0.883 |
| DES use | 138 | (92.0%) | 134 | (89.3%) | 0.427 |
| Number of stents used (mean ± | 1.30 ± 0.52 | | 1.39 ± 0.71 | | 0.244 |
| SD) | | | | | |
| Stent Type | | | | | |
| Xience | 104 | (69.3%) | 111 | (74.0%) | 0.596 |
| Resolute Integrity | 6 | (4.0%) | 3 | (2.0%) | 0.580 |
| Promus Premier | 13 | (8.7%) | 9 | (6.0%) | 0.651 |
| Biofreedom | 1 | (0.7%) | 1 | (0.7%) | 0.985 |
| Synergy | 5 | (3.3%) | 7 | (4.7%) | 0.834 |
| Biomatrix | 1 | (0.7%) | 0 | | 0.594 |
| Stent length (mm, mean ± SD) | | | | | |
| First Stent | 26.88 ± 9.96 | | 27.36 ± 10.26 | | 0.698 |
| Second Stent | 21.79 ± 8.51 | | 21.94 ± 11.29 | | 0.951 |
| Third Stent | 17.30 ± 7.38 | | 16.20 ± 7.16 | | 0.788 |
| Fourth Stent | 26.50 ± 16.63 | | | | |
| Stent diameter | | | | | |
| (mm, mean ± SD) | | | | | |
| First Stent | 3.13 ± 0.46 | | 3.10 ± 0.46 | | 0.590 |
| Second Stent | 3.26 ± 0.48 | | 3.64 ± 2.98 | | 0.434 |
| Third Stent | 3.25 ± 0.49 | | 3.60 ± 0.42 | | 0.194 |
| Fourth Stent | 3.00 ± 0.71 | | | | |
| Procedural Success | 150 | (100%) | 150 | (100%) | — |

TABLE 4
All quantitative assessments were made using quantitative coronary angiography.
Data are shown as mean +/− SEM. Data demonstrates a reduction in
late lumen loss in patients receiving dietary nitrate for 6 months associated
with statistically significant improvements in flow and lumen diameter.

| | Intervention | | |
|---|---|---|---|
| | (n = 99) | Placebo (n = 89) | P value |
| Baseline stent diameter (mm) | 3.206 ± 0.047 | 3.146 ± 0.043 | 0.356 |
| Baseline stent length (mm) | 27.92 ± 1.257 | 27.16 ± 1.380 | 0.685 |
| Baseline proximal reference stent diameter (mm) | 3.387 ± 0.065 | 3.519 ± 0.170 | 0.452 |
| Baseline distal reference stent diameter (mm) | 2.685 ± 0.054 | 2.675 ± 0.050 | 0.886 |
| Baseline stent MLD (mm) | 2.720 ± 0.049 | 2.679 ± 0.042 | 0.531 |
| Baseline stent diameter stenosis (%) | 12.43 ± 0.682 | 12.94 ± 0.768 | 0.616 |
| Baseline proximal maximum segment diameter | 3.788 ± 0.078 | 3.821 ± 0.118 | 0.813 |
| (mm) | | | |
| Baseline distal maximum segment diameter | 2.974 ± 0.076 | 3.090 ± 0.074 | 0.278 |
| (mm) | | | |
| Baseline segment MLD (mm) | 2.448 ± 0.056 | 2.418 ± 0.046 | 0.683 |
| Baseline segment diameter stenosis (%) | 18.37 ± 1.025 | 18.56 ± 0.869 | 0.888 |
| Flow up stent MLD (mm) | 2.606 ± 0.056 | 2.436 ± 0.046 | 0.0222 |
| Follow up diameter stenosis stent (%) | 15.43 ± 0.966 | 15.16 ± 1.015 | 0.851 |
| Follow up segment MLD (mm) | 2.393 ± 0.062 | 2.147 ± 0.049 | 0.0024 |
| Follow up diameter stenosis segment (%) | 18.63 ± 1.029 | 19.36 ± 1.095 | 0.631 |
| Stent late lumen loss (mm) | 0.117 ± 0.038 | 0.244 ± 0.036 | 0.0165 |
| Segment late lumen loss (mm) | 0.055 ± 0.050 | 0.269 ± 0.039 | 0.0011 |
| Stent stenosis change (%) | 3.105 ± 1.077 | 2.257 ± 0.833 | 0.541 |
| Segment stenosis change (%) | 0.346 ± 1.339 | 0.872 ± 1.236 | 0.775 |

TABLE 5
Change from baseline at 6 months in blood pressure, heart rate
and methaemaglobinaemia levels in the intervention arm and the
placebo arm are shown (BP, blood pressure; BPM, beats per minute).
Statistical significance determined using unpaired t-test.

| | Intervention | Placebo | |
|---|---|---|---|
| | (n = 300) | (n = 300) | P value |
| Heart rate (BPM) (Mean ± SD) | −4.10 ± 16.36 | −0.60 ± 10.05 | 0.060 |
| Systolic BP (mmHg) (Mean ± SD) | −11.99 ± 15.93 | 1.51 ± 17.49 | <0.0001 |
| Diastolic BP (mmHg) (Mean ± SD) | −5.96 ± 9.71 | −0.09 ± 10.50 | <0.0001 |
| Methaemaglobinanemia level (Mean ± SD) | 0.42 ± 0.36 | −0.02 ± 0.22 | <0.0001 |

1. Inorganic nitrate for use in a method of preventing restenosis and/or device thrombosis in a patient undergoing percutaneous coronary intervention (PCI), non-coronary percutaneous intervention and/or coronary artery bypass grafting (CABG), said method comprising administering to said patient 2-12 mmol of an inorganic nitrate once per day after said PCI, non-coronary percutaneous intervention and/or CABG.

2. Inorganic nitrate for use according to claim 1, wherein the inorganic nitrate is administered orally.

3. Inorganic nitrate for use according to claim 2, wherein the inorganic nitrate is dietary nitrate.

4. Inorganic nitrate for use according to claim 3, wherein the dietary nitrate is administered in the form of beetroot, green leafy vegetables or any other nitrate-rich vegetable or fruit or the juice thereof.

5. Inorganic nitrate for use according to claim 2, wherein the inorganic nitrate is a nitrate salt.

6. Inorganic nitrate for use according to claim 5, wherein the nitrate salt is potassium nitrate (KNO₃) or sodium nitrate (NaNO₃).

7. Inorganic nitrate for use according to claim 5 or 6, wherein the inorganic nitrate is administered in the form of a capsule, pill, lozenge, spray, toothpaste or chewing gum.

8. Inorganic nitrate for use according to any one of the preceding claims, wherein 2.5-8 mmol of inorganic nitrate is administered.

9. Inorganic nitrate for use according to claim 8, wherein 4-5 mmol of inorganic nitrate is administered.

10. Inorganic nitrate for use according to any one of the preceding claims, wherein the inorganic nitrate is administered in combination with an antioxidant.

11. Inorganic nitrate for use according to any one of the preceding claims, wherein inorganic nitrite is administered from a coating applied to a stent or scaffold or prosthesis used in said PCI or non-coronary percutaneous intervention.

12. Inorganic nitrate for use according to any one of the preceding claims, wherein the inorganic nitrate is administered prior to said PCI, non-coronary percutaneous intervention and/or CABG.

13. Inorganic nitrate for use according to claim 12, wherein the inorganic nitrate is administered at least 2 hours prior to said PCI, non-coronary percutaneous intervention and/or CABG.

14. Inorganic nitrate for use according to claim 13, wherein the inorganic nitrate is administered no more than 24 hours prior to said PCI, non-coronary percutaneous intervention and/or CABG.

15. Inorganic nitrate for use according to any one of the preceding claims, wherein the inorganic nitrate is administered for 1-5 months after said PCI, non-coronary percutaneous intervention and/or CABG.

16. Inorganic nitrate for use according to any one of claims 1 to 14, wherein the inorganic nitrate is administered for at least 6 months after said PCI, non-coronary percutaneous intervention and/or CABG.

17. Inorganic nitrate for use according to claim 15, wherein the inorganic nitrate is administered indefinitely.

18. Inorganic nitrate for use according to any one of the preceding claims, wherein the patient has stable angina or an acute coronary syndrome.

19. Inorganic nitrate for use according to claim 18, wherein the acute coronary syndrome is unstable angina or myocardial infarction.

20. Inorganic nitrate for use according to any one of the preceding claims, wherein the patient has diabetes.

21. Inorganic nitrate for use according to any one of the preceding claims, wherein the patient has peripheral vascular disease.

22. Inorganic nitrate for use according to any one of the preceding claims, wherein administration of the inorganic nitrate reduces major adverse cardiac events (MACE).
